(19) United States
(12) Patent Application Publication
(10) Pub. No.: US 2022/0036223 A1
(43) Pub. Date: Feb. 3, 2022

(54) PROCESSING APPARATUS, PROCESSING METHOD, AND NON-TRANSITORY STORAGE MEDIUM

(71) Applicant: NEC Corporation, Minato-ku, Tokyo (JP)

(72) Inventor: Riki ETO, Tokyo (JP)

(73) Assignee: NEC Corporation, Minato-ku, Tokyo (JP)

(21) Appl. No.: 17/279,315

(22) PCT Filed: Sep. 27, 2018
(86) PCT No.: PCT/JP2018/035849

§ 371 Date: Mar. 24, 2021

(51) Int. Cl. G06N 7/00; G06N 20/00; G01N 33/00

(52) U.S. Cl. CPC G06N 7/00; G01N 33/0036; G06N 20/00

(57) ABSTRACT

A processing apparatus (20) includes a prediction equation generation unit (210) and an output unit (250). The prediction equation generation unit (210) generates, through machine learning having a plurality of feature values based on outputs from a set of a plurality of kinds of sensors and correct answer data as inputs, a prediction equation that has the plurality of feature values as variables and is used for predicting an odor component. The output unit (250) outputs a plurality of weights as information indicating the prediction equation in association with the feature values, respectively.

### TECHNICAL FIELD

The present invention relates to a processing apparatus, a processing method, and a program.

### BACKGROUND ART

A technique that obtains information regarding gas by measuring gas with a sensor has been developed.

Patent Document 1 discloses an odor sensor in which a plurality of sensor elements are provided. Specifically, a configuration in which a plurality of sensor elements are provided with substance absorption membranes having different characteristics, respectively, and each sensor element exhibits a reaction specific to a molecule to which the action of the sensor is directed.

### RELATED DOCUMENT

### Patent Document

[Patent Document 1] International Publication No. WO2017/085939

### SUMMARY OF THE INVENTION

### Technical Problem

However, Patent Document 1 does not disclose how a combination of sensor elements should be selected according to the purpose of detection.

The invention has been accomplished in view of the above-described problem. An object of the invention is to provide a technique that derives an appropriate combination of sensors for a desired purpose.

### Solution to Problem

A first processing apparatus of the invention includes:
a prediction equation generation unit that generates, through machine learning having a plurality of feature values based on outputs from a set of a plurality of kinds of sensors and correct answer data as inputs, a prediction equation that has the plurality of feature values as variables and is used for predicting an odor component;
an extraction unit that extracts one or more sensors from the set based on a plurality of weights to the plurality of feature values in the prediction equation; and
an output unit that outputs at least one of the sensors extracted by the extraction unit and the unextracted sensors in an identifiable state,
in which the extraction unit extracts the sensors that are output sources of the feature values weighted with the weights satisfying or not satisfying a predetermined condition among the plurality of weights in the prediction equation.

A second processing apparatus of the invention includes:
a prediction equation generation unit that generates, through machine learning having a plurality of feature values based on outputs from a set of a plurality of kinds of sensors and correct answer data as inputs, a prediction equation that has the plurality of feature values as variables and is used for predicting an odor component;
an output unit that outputs a plurality of weights to the plurality of feature values in the prediction equation as information indicating the prediction equation in association with the feature values, respectively.

A first processing method of the invention includes:
a prediction equation generation step of generating, through machine learning having a plurality of feature values based on outputs from a set of a plurality of kinds of sensors and correct answer data as inputs, a prediction equation that has the plurality of feature values as variables and is used for predicting an odor component;
an extraction step of extracting one or more sensors from the set based on a plurality of weights to the plurality of feature values in the prediction equation; and
an output step of outputting at least one of the sensors extracted in the extraction step and the unextracted sensors in an identifiable state,
in which, in the extraction step, the sensors that are output sources of the feature values weighted with the weights satisfying or not satisfying a predetermined condition among the plurality of weights in the prediction equation are extracted.

A second processing method of the invention includes:
a prediction equation generation step of generating, through machine learning having a plurality of feature values based on outputs from a set of a plurality of kinds of sensors and correct answer data as inputs, a prediction equation that has the plurality of feature values as variables and is used for predicting an odor component;
an output step of outputting a plurality of weights to the plurality of feature values in the prediction equation as information indicating the prediction equation in association with the feature values, respectively.

A program of the invention
causes a computer to execute each step of the processing method of the invention.

### Advantageous Effects of Invention

According to the invention, it is possible to provide a technique that derives an appropriate combination of sensors for a desired purpose.

### BRIEF DESCRIPTION OF THE DRAWINGS

The above-described object and other objects, features, and advantages will become apparent from preferable example embodiments described below and the accompanying drawings.

FIG. 1 is a diagram illustrating the configuration of a processing apparatus according to a first example embodiment.

FIG. 2 is a diagram illustrating a sensor.

FIG. 3 is a diagram illustrating time-series data.

FIG. 4 is a diagram illustrating sensor output data from a set of a plurality of kinds of sensors.

FIG. 5 is a flowchart illustrating a processing method according to the first example embodiment.

FIG. 6 is a diagram illustrating a computer for implementing the processing apparatus.

FIG. 7 is a diagram illustrating the configuration of a processing apparatus according to a second example embodiment.

FIG. 8 is a flowchart illustrating a processing method according to the second example embodiment.

FIG. 9 is a diagram illustrating a prediction model that is used in machine learning to be performed by a prediction equation generation unit according to a third example embodiment.

FIG. 10 is a diagram illustrating the configuration of a processing apparatus according to a fourth example embodiment.

FIG. 11 is a flowchart illustrating a processing method according to the fourth example embodiment.

### DESCRIPTION OF EMBODIMENTS

Hereinafter, example embodiments of the invention will be described referring to the drawings. In all drawings, the same components are represented by the same reference numerals, and description thereof will not be repeated.

Note that, in the following description, except for a case where particular description is provided, each component of each apparatus is not a configuration of a hardware unit but a configuration of a function unit. Each component of each apparatus is implemented by any combination of hardware and software centering on a CPU, a memory, a program loaded to the memory to implement the components in the drawings, a storage medium, such as a hard disk, storing the program, and an interface for network connection of any computer. Then, there are various modification examples of the implementation method and apparatus.

### First Example Embodiment

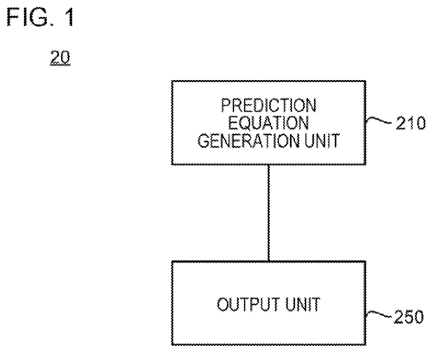

FIG. 1 is a diagram illustrating the configuration of a processing apparatus 20 according to a first example embodiment. The processing apparatus 20 according to the example embodiment includes a prediction equation generation unit 210 and an output unit 250. The prediction equation generation unit 210 generates, through machine learning having a plurality of feature values based on outputs of a set of a plurality of kinds of sensors and correct answer data as inputs, a prediction equation that has the plurality of feature values as variables and is used for predicting an odor component. The output unit 250 outputs a plurality of weights to a plurality of feature values in the prediction equation as information indicating the prediction equation in association with the feature values, respectively. Details will be described below.

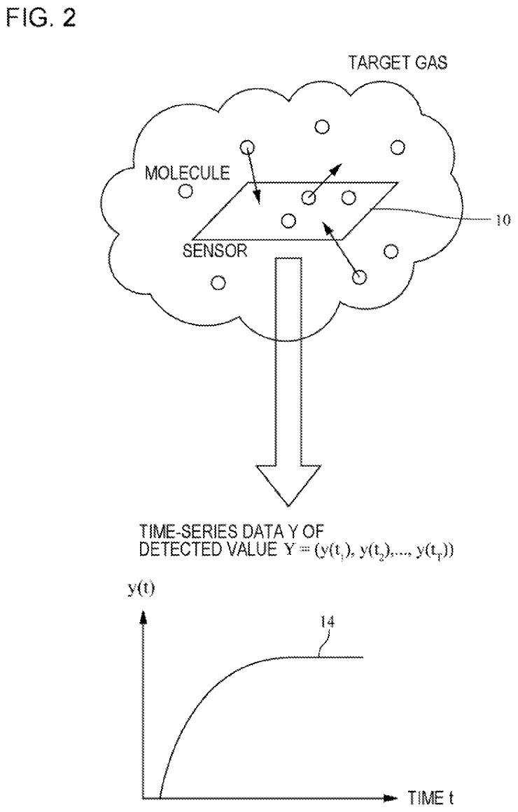

FIG. 2 is a diagram illustrating a sensor 10. The sensor 10 is a sensor that has a receptor to which a molecule is attached, and of which a detected value changes according to attachment and detachment of the molecule to and from the receptor. Note that gas that is sensed by the sensor 10 is referred to as target gas. Furthermore, time-series data of the detected value output from the sensor 10 is referred to as time-series data 14. Here, as needed, time-series data 14 is also denoted as Y, and the detected value at a time t is also denoted as y(t). Y is a vector in which y(t) is listed.

For example, the sensor 10 is a membrane-type surface stress sensor (MSS). The MSS has a functional membrane, to which a molecule is attached, as a receptor, and stress that occurs in a support member of the functional membrane changes depending on attachment and detachment of the molecule to and from the functional membrane. The MSS outputs a detected value based on the change in stress.

Various materials, such as organic materials, inorganic-materials, and biological materials, can be used for the functional membrane of the MSS. A response target molecule and a response characteristic of the sensor 10 depend on the functional membrane. Accordingly, it is possible to analyze complicated odor composed of mixed gas including various components by combining a plurality of kinds of sensors 10 having different functional membranes.

Note that the sensor 10 is not limited to the MSS, any sensor may be applied as long as a sensor outputs a detected value based on change in physical quantity related to viscoelasticity or a dynamic characteristic (mass, inertial moment, or the like) of a member of the sensor 10 caused by attachment and detachment of a molecule to and from the receptor, and various types of sensors, such as a cantilever type, a membrane type, an optical type, a piezoelectric type, and a vibration response type, can be employed. In such sensors 10, it is possible to combine a plurality of kinds of sensors 10 that are different in at least one of the response target molecule and the response characteristic of the sensor 10.

Here, there are many kinds of sensors 10. On the other hand, there is a limit to the number of sensors 10 that can be actually used in a detection apparatus. Accordingly, there is a need to select what kinds of sensors 10 should be used in combination for detection according to the purpose.

In the example embodiment, the prediction equation generation unit 210 generates a prediction equation for predicting an odor component through machine learning having a plurality of feature values based on outputs from a set of a plurality of kinds of sensors 10 and correct answer data as inputs. The prediction equation is an equation that has a plurality of feature values as variables, and a weight to each feature value in the prediction equation corresponds to the magnitude of contribution of the feature value to a prediction result. Accordingly, it is possible to discriminate the sensor 10 with large contribution to the purpose and the sensor 10 with small contribution to the purpose based on information indicating the prediction equation.

The feature value and the prediction equation will be described below in detail. The feature value is a value that is obtained based on the output of the sensor 10. Note that one or more feature values are obtained for one sensor 10, and each feature value depends only on the output of one sensor 10.

The time-series data 14 is time-series data in which the detected values output from the sensor 10 are arranged in an ascending order of an output time from the sensor 10. Note that the time-series data 14 may be obtained by executing predetermined preprocessing on the time-series data of the detected values obtained from the sensor 10. As the preprocessing, for example, filtering for removing a noise component from the time-series data, or the like can be employed.

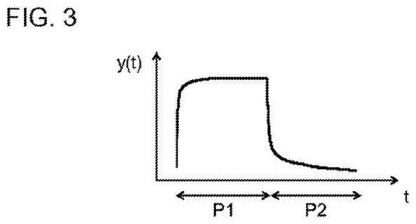

FIG. 3 is a diagram illustrating the time-series data 14. The time-series data 14 is obtained by exposing the sensor 10 to the target gas. Note that the time-series data 14 may be obtained by an operation to expose the sensor 10 to gas to be measured and an operation to remove gas to be measured from the sensor 10. In the example of the drawing, data of a period P1 is obtained by exposing the sensor 10 to the target gas, and data of a period P2 is obtained by an operation to remove the gas to be measured from the sensor 10. Note that examples of the operation to remove the gas to be measured from the sensor 10 include an operation to expose the sensor 10 to the purge gas. Furthermore, in a measurement of the target gas by the sensor 10, the operation to expose the sensor 10 to the gas to be measured and the operation to remove the gas to be measured from the sensor 10 may be repeatedly performed to obtain a plurality of pieces of time-series data 14.

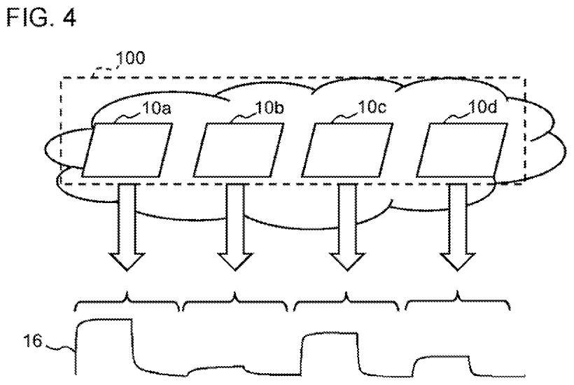

FIG. 4 is a diagram illustrating sensor output data 16 from a set 100 of a plurality of kinds of sensors 10. In an example of the drawing, the set 100 of the sensors 10 includes a first sensor 10a, a second sensor 10b, a third sensor 10c, and a fourth sensor 10d. For example, the set 100 is modularized, and a measurement is performed for the same target gas in the same detection environment. The set 100 of the sensors 10 includes a plurality of sensors 10 freely chosen from a large number of usable sensors 10. The sensor output data 16 is data obtained by combining the time-series data 14 obtained from a plurality of kinds of respective sensors 10. In the example of the drawing, the sensor output data 16 is obtained by arranging the time-series data 14 of the first sensor 10a, the second sensor 10b, the third sensor 10c, and the fourth sensor 10d in order.

A plurality of feature values can be computed from the sensor output data 16. Here, it is assumed that a feature value vector X is a vector having a plurality of feature values as elements. In the feature value vector X, a plurality of feature values xⱼ (j=1, 2, . . . , J) based on outputs of a plurality of kinds of sensors 10 included in the set 100 are included. Note that xⱼ may be a numerical value or may be a vector. In a case where xⱼ is a vector, xⱼ is a vector having a plurality of feature values based on the output of the same sensor 10 as elements. The feature value xⱼ is, for example, the time-series data 14 of the sensor 10, data obtained by differentiating the time-series data 14, or a set Ξ of contribution values described below. The prediction equation generation unit 210 can acquire the time-series data 14 or the sensor output data 16 and can compute the feature values based on the acquired data. Note that the prediction equation generation unit 210 may acquire the feature values derived outside the processing apparatus 20 instead of acquiring the time-series data 14 or the sensor output data 16.

The prediction equation is a linear sum of the feature values, and is represented by z=WX+b. Here, W is a vector, and b is a constant. Then, each element of a weight W is a coefficient to each element of the feature value vector X. Then, z to be obtained indicates a prediction result. The prediction equation may be used in discrimination or may be used in regressive prediction. For example, in a prediction equation that is used in discrimination of the presence or absence of a certain odor component, in a case where z is equal to or greater than a predetermined reference, determination is made that the odor component to be detected is included in the gas to be measured, and in a case where z is less than the reference, determination is made that the odor component to be detected is not included in the gas to be measured. Examples of the regressive prediction include prediction of manufacturing quality based on odor of a product, such as a drink, prediction of an in-vivo state by a measurement of expiration, and the like.

Note that the forms of the time-series data 14, the sensor output data 16, the feature values, and the prediction equation are examples, and the time-series data 14, the sensor output data 16, the feature values, and the prediction equation according to the example embodiment are not limited to the above-described forms.

The set Ξ of the contribution values as an example of the feature values will be described below. Here, for description, sensing by the sensor 10 is modeled as follows.
(1) The sensor 10 is exposed to the target gas including K kinds of molecules.
(2) A concentration of each molecule k in the target gas is fixed ρₖ.
(3) N molecules in total can be absorbed to the sensor 10.
(4) The number of molecules k attached to the sensor 10 at a time t is nₖ(t).

A temporal change of the number nₖ(t) of molecules k attached to the sensor 10 is formulated as follows.

$$\frac{d {n}_{k} ( t )}{d t} = {α}_{k} {ρ}_{k} - {β}_{k} {n}_{k} ( t ) \qquad ( 1 )$$

A first term and a second term of a right side of Equation (1) represent an increase amount (the number of molecules k newly attached to the sensor 10) and a decrease amount (the number of molecules k detached from the sensor 10) of the molecules k per unit time, respectively. Furthermore, αₖ and βₖ are a velocity constant representing a velocity at which the molecules k are attached to the sensor 10 and a velocity constant representing a velocity at which the molecules k are detached from the sensor 10, respectively.

Here, since the concentration ρₖ is fixed, the number nₖ(t) of molecules k at the time t can be formulated as follows from Foliation (1) described above.

$${n}_{k} ( t ) = {n}_{k}^{*} + ( {n}_{k} ( {t}_{0} ) - {n}_{k}^{*} ) {e}^{- {β}_{k} t} \\ where {n}_{k}^{*} := \frac{{β}_{k} {ρ}_{k}}{{α}_{k}} \qquad ( 2 )$$

In a case where it is assumed that no molecules are attached to the sensor 10 at a time to (initial state), nₖ(t) is represented as follows.

nₖ(t)=nₖ*(1−e^{-β}^{ₖ}^{t}) (3)

The detected value of the sensor 10 is determined based on stress acting on the sensor 10 due to the molecules included in the target gas. Then, it is considered that the stress acting on the sensor 10 due to a plurality of molecules can be represented by a linear sum of stress acting on the individual molecules. Note that it is considered that stress caused by the molecule is different depending on the kind of the molecule. That is, it can be said that the contribution of the molecule to the detected value of the sensor 10 is different depending on the kind of the molecule.

Accordingly, the detected value y(t) of the sensor 10 can be formulated as follows.

$$\begin{matrix}y ( t ) = {\sum }_{k = 1}^{K} {γ}_{k} {n}_{k} ( t ) \\ = \{ \begin{matrix}{ξ}_{0} - {\sum }_{k = 1}^{K} {ξ}_{k} {e}^{- {β}_{k} t} & ( case of rise ) \\ {\sum }_{k = 1}^{K} {ξ}_{k} {e}^{- {β}_{k} t} & ( case of fall )\end{matrix}\end{matrix} \\ where {ξ}_{k} = \frac{{γ}_{k} {α}_{k} {ρ}_{k}}{{β}_{k}} ( k = 1 , … , K ) , {ξ}_{0} = {\sum }_{k = 1}^{K} {ξ}_{k} \qquad ( 4 )$$

Here, both γₖ and ξₖ represent the contribution of the molecule k to the detected value of the sensor 10. Note that a “rise” corresponds to the above-described period P1, and a “fall” corresponds to the above-described period P2.

Here, in a case where the time-series data 14 obtained from the sensor 10 that senses the target gas can be resolved as Equation (4) described above, it is possible to recognize the kind of the molecules included in the target gas or proportions of various kinds of molecules included in the target gas. That is, data (that is, the feature values of the target gas) representing the features of the target gas is obtained by the resolution shown in Equation (4).

Accordingly, the time-series data 14 output from the sensor 10 is resolved as shown in Equation (5) described below using a set Θ={θ₁, θ₂, . . . , θₘ} of feature constants. Note that the set Θ of the feature constants may be determined in advance or may be generated by the processing apparatus 20.

$$y ( t ) = {\sum }_{i = 1}^{m} {ξ}_{i} f ( {θ}_{i} ) \qquad ( 5 )$$

Here, ξᵢ is a contribution value representing contribution of a feature constant θᵢ to the detected value of the sensor 10.

With such resolution, the contribution value ξᵢ representing the contribution of each feature constant θᵢ to the time-series data 14 is computed. The set Ξ of the contribution values ξᵢ can be set as the feature values representing the features of the target gas. The set of the contribution values ξᵢ Dis represented by, for example, a feature vector Ξ=(ξ₁, ξ₂, . . . , ξₘ) in which ξᵢ is listed. Note that it is not the case that the feature values of the target gas should be expressed as a vector.

Here, as the feature constant θ, the above-described velocity constant β or a time constant τ that is a reciprocal of the velocity constant can be employed. In regard to cases where β and τ are used as θ, Equation (5) can be represented as follows.

$$\begin{matrix}y ( t ) = {\sum }_{i = 1}^{m} {ξ}_{i} {e}^{- {β}_{i} t} \\ & ( 6 ) \\ y ( r ) = {\sum }_{i = 1}^{m} {ξ}_{i} {e}^{- t / {τ}_{i}} & ( 7 )\end{matrix}$$

As described above, since it is considered that the contribution of the molecule to the detected value of the sensor 10 is different depending on the kind of the molecule, it is considered that the set Ξ of the contribution values described above is different according to the kind of the molecule included in the target gas or a blend ratio of the molecule. Therefore, the set Ξ of the contribution values can be used as information capable of distinguishing gas in which a plurality of kinds of molecules are mixed, that is, the feature values of the gas.

In a case where the set Ξ of the contribution values is used as the feature values of the target gas, there is an advantage other than an advantage that gas including a plurality of kinds of molecules is handled. First, there is an advantage that the degree of similarity of gas can be easily recognized. For example, in a case where the feature values of the target gas are expressed by vectors, the degree of similarity of gas can be easily recognized based on a distance between the feature vectors.

Furthermore, in a case where the set Ξ of the contribution values is set as the feature values, there is an advantage that robustness can be achieved regarding change in time constant to change in blend ratio or change in blend ratio. The term “robustness” used herein is a property that the feature value to be obtained also changes slightly when a measurement environment or a measurement target changes slightly.

In a case where robustness is achieved for the change in blend ratio, for example, in a case where a blend ratio of gas is changing gradually regarding mixed gas in which two kinds of gas are mixed, the feature value is also changing gradually. It can be understood that this property appears since the contribution value ξₖ is proportional to ρₖ representing the concentration of the gas in Equation (4), and accordingly, small change in concentration appears as small change in contribution value.

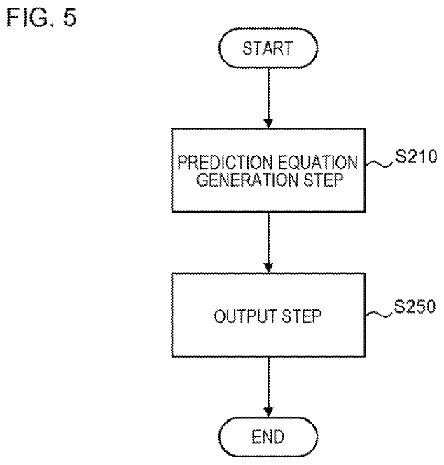

FIG. 5 is a flowchart illustrating a processing method according to the first example embodiment. The processing method according to the example embodiment includes a prediction equation generation step S210 and an output step S250. In the prediction equation generation step S210, through machine learning having a plurality of feature values based on outputs from a set 100 of a plurality of kinds of sensors 10 and correct answer data as inputs, a prediction equation that has a plurality of feature values as variables and is used for predicting an odor component is generated. In the output step S250, a plurality of weights to a plurality of feature values in the prediction equation are output as information indicating the prediction equation in association with the feature values, respectively. The processing method according to the example embodiment is implemented by the processing apparatus 20. Details will be described below.

The prediction equation generation unit 210 acquires the time-series data 14, the sensor output data 16, or the feature value vector X. The prediction equation generation unit 210 may acquire the time-series data 14, the sensor output data 16, or the feature value vector X from a storage apparatus accessible from the prediction equation generation unit 210, may be acquired from an apparatus outside the processing apparatus 20, or may be acquired from the sensor 10. The feature value vector X may be obtained by a measurement in a certain situation or may be prepared in advance and held in a storage apparatus. Furthermore, the prediction equation generation unit 210 acquires correct answer data to the feature value vector X. The correct answer data may be input to the processing apparatus 20 by a user or may be stored in the storage apparatus accessible from the prediction equation generation unit 210 in association with the feature value vector (that is, a plurality of feature values) in advance.

Then, the prediction equation generation unit 210 generates the prediction equation through the machine learning having a plurality of feature values and the correct answer data as inputs in the prediction equation generation step S210. Specifically, the prediction equation generation unit 210 derives the weight W and the constant b. A plurality of feature values are, for example, the above-described feature value vector X. A plurality of feature values are obtained by a measurement result of known target gas with the set 100 of the sensors 10. Then, the correct answer data is information indicating a prediction result that should be obtained for the feature value vector with the prediction equation. That is, the correct answer data is information corresponding to the measured known target gas.

Here, the prediction equation generation unit 210 can increase the accuracy of the prediction equation through machine learning using a plurality of pieces of learning data sets including a plurality of feature values and correct answer data. As described above, a plurality of learning data sets are obtained by repeatedly performing the operation to expose the sensor 10 to the gas to be measured and operation to remove the gas to be measured from the sensor 10 in the measurement of the target gas by the sensor 10. The prediction equation generation unit 210 ends learning, for example, in a case where the number of repetitions of learning (the number of learning data sets) determined in advance is satisfied. Note that it is preferable that the detection environment by the set 100 of the sensors 10 is identical in the plurality of learning data sets. Furthermore, it is preferable that the detection environment is similar to a detection environment when the sensors 10 and the generated prediction equation are actually used.

Note that the feature value to be used in the machine learning may be obtained by simulating a response of the sensor 10 to the target gas. Note that a plurality of learning data sets may be generated using results obtained under simulation conditions that the detection environment are different each other. Note that, in a case where a plurality of different simulation results are obtained for the same detection environment, a plurality of learning data sets may be generated using results under the same simulation condition.

Next, the output unit 250 according to the example embodiment outputs information indicating the prediction equation generated in the prediction equation generation unit 210 in the output step S250. Specifically, the output unit 250 outputs a plurality of weights and the constant b as information indicating the prediction equation in association with the feature values, respectively. For example, each value of the weights is displayed on a display apparatus in a state in which the feature value of the sensor 10 in the set 100 of the sensors 10, to which the value corresponds, is understandable. Then, the user can recognize the degree of contribution of each sensor 10 to the prediction result by confirming the weight to each feature value. Then, the user can exchange, for example, the sensor 10 having low contribution to the prediction result among the sensors 10 for another kind of sensor 10. Note that, in a case where a plurality of feature value and weights are present for each sensor 10, the user can regard the sensor 10, most of a plurality of weights of which is zero, as the sensor 10 having low contribution to the prediction result.

In a case where the user of the processing apparatus 20 attempts to manufacture a sensor module including a plurality of sensors 10, for example, for a specific purpose, the user of the processing apparatus 20 uses the processing apparatus 20 in selecting the sensors 10 to be included in the sensor module. An upper limit of the number of sensors 10 to be included in the set 100 of the sensors 10 is determined by the number of sensors 10 mountable on the sensor module. For example, the user exchanges the sensor 10 having low contribution to the prediction result based on an output of the processing apparatus 20 for another usable kind of sensor 10, and operates the processing apparatus 20 in the same manner again. Then, the exchange of the sensor 10 and the operation of the processing apparatus 20 are repeatedly performed until all the sensors 10 mounted on the sensor module are brought into a state of sufficiently contributing to the prediction result. In this manner, it is possible to obtain a combination of the sensors 10 capable of accomplishing a desired purpose with a limited number of sensors 10.

In addition, the user can predict an odor component using a combination of the sensors 10 finally employed and a prediction equation generated for the combination. Specifically, in predicting the odor component, the feature values are computed based on the outputs from a plurality of sensors 10, and the feature values are applied to the prediction equation. Then, a prediction result is obtained based on a computed value by the prediction equation.

Note that the output unit 250 may output information indicating the prediction equation to an external apparatus or may store the above-described information in a storage apparatus accessible from the output unit 250.

Each functional component of the processing apparatus 20 may be implemented by hardware (for example, a hard-wired electronic circuit or the like) that implements each functional component or may be implemented by a combination of hardware and software (for example, a combination of an electronic circuit and a program that controls the electronic circuit, or the like). Hereinafter, a case where each functional component of the processing apparatus 20 is implemented by a combination of hardware and software will be further described.

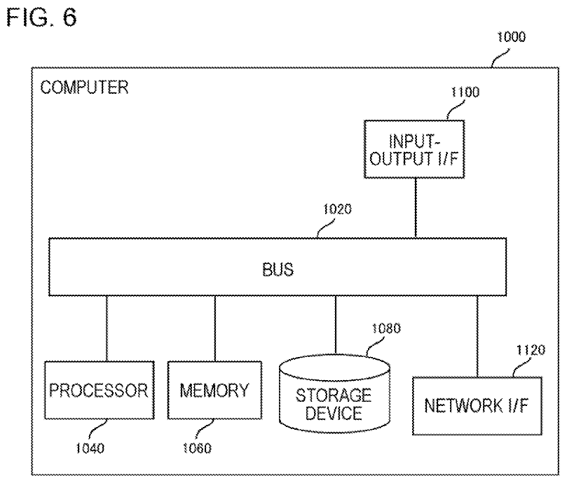

FIG. 6 is a diagram illustrating a computer 1000 for implementing the processing apparatus 20. The computer 1000 is any computer. For example, the computer 1000 is a stationary computer, such as a personal computer (PC) or a server machine. In addition, for example, the computer 1000 is a portable computer, such as a smartphone or a tablet terminal. The computer 1000 may be a dedicated computer designed to implement the processing apparatus 20 or may be a general-purpose computer.

The computer 1000 has a bus 1020, a processor 1040, a memory 1060, a storage device 1080, an input-output interface 1100, and a network interface 1120. The bus 1020 is a data transmission path through which the processor 1040, the memory 1060, the storage device 1080, the input-output interface 1100, and the network interface 1120 transmit and receive data from one another. Note that a method of connecting the processor 1040 and the like to one another is not limited to bus connection.

The processor 1040 is various processors, such as a central processing unit (CPU), a graphics processing unit (GPU), and a field-programmable gate array (FPGA). The memory 1060 is a main storage apparatus that is implemented using a random access memory (RAM) or the like. The storage device 1080 is an auxiliary storage apparatus that is implemented using a hard disk, a solid state drive (SSD), a memory card, or a read only memory (ROM).

The input-output interface 1100 is an interface that connects the computer 1000 and an input-output device. For example, an input apparatus, such as a keyboard, and an output apparatus, such as a display apparatus, are connected to the input-output interface 1100. In addition, for example, the sensor 10 is connected to the input-output interface 1100. Note that the sensor 10 is not necessarily connected directly to the computer 1000. For example, the sensor 10 may store the time-series data 14 in a storage apparatus that is shared with the computer 1000.

The network interface 1120 is an interface that connects the computer 1000 to a communication network. The communication network is, for example, a local area network (LAN) or a wide area network (WAN). A method in which the network interface 1120 is connected to the communication network may be wireless connection or may be wired connection.

The storage device 1080 stores a program module that implements each functional component of the processing apparatus 20. The processor 1040 reads each program module to the memory 1060 and executes each program module, thereby implementing a function corresponding to each program module.

Next, the operations and effects of the example embodiment will be described. With the processing apparatus 20 according to the example embodiment, the degree of contribution of each sensor 10 to the prediction result can be recognized based on information indicating the prediction equation. Consequently, an appropriate combination of the sensors can be derived for a desired purpose.

### Second Example Embodiment

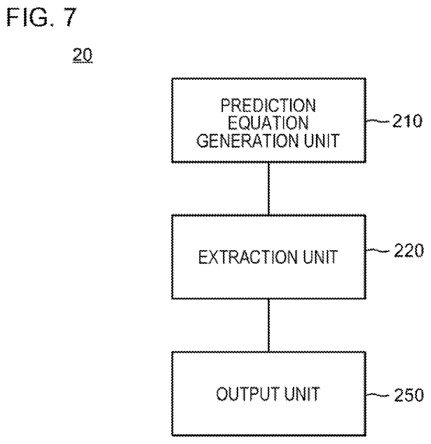

FIG. 7 is a diagram illustrating the configuration of a processing apparatus 20 according to a second example embodiment. The processing apparatus 20 according to the example embodiment is the same as the processing apparatus 20 according to the first example embodiment except for the following matters.

The processing apparatus 20 according to the example embodiment further includes an extraction unit 220. The extraction unit 220 extracts one or more sensors 10 from the set 100 based on a plurality of weights to a plurality of feature values in the prediction equation. Specifically, the extraction unit 220 extracts the sensors 10 that are output sources of the feature values weighted with the weights satisfying or not satisfying a predetermined condition among a plurality of weights in the prediction equation.

Furthermore, in the example embodiment, the output unit 250 outputs at least one of the sensors 10 extracted by the extraction unit 220 and the unextracted sensors 10 in an identifiable state. Note that, in the example embodiment, the output unit 250 does not necessarily output information indicating the prediction equation. Details will be described below.

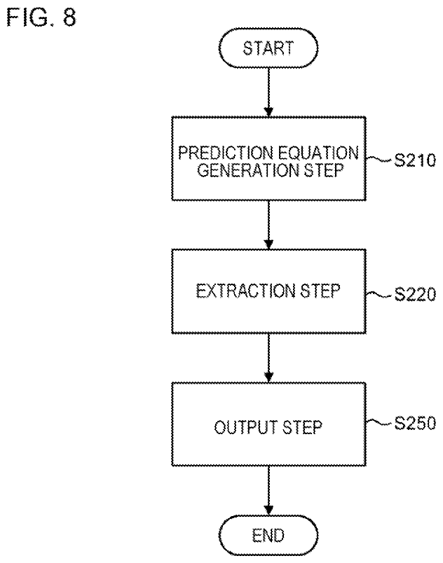

FIG. 8 is a flowchart illustrating a processing method according to the second example embodiment. The processing method according to the example embodiment is the same as the processing method according to the first example embodiment except that, the processing method further includes an extraction step S220, and in the output step S250, at least one of the sensors 10 extracted in the extraction step S220 and the unextracted sensors 10 is output in an identifiable state. In the extraction step S220, one or more sensors 10 are extracted from the set 100 based on a plurality of weights to a plurality of feature values in the prediction equation. Specifically, in the extraction step S220, the sensors 10 that are output sources of the feature values weighted with the weights satisfying or not satisfying a predetermined condition among a plurality of weights in the prediction equation are extracted.

The processing method is implemented by the processing apparatus 20 according to the example embodiment. The operation of the processing apparatus 20 will be described below in detail.

In the example embodiment, the prediction equation generation step S210 is the same as the prediction equation generation step S210 according to the first example embodiment. In the example embodiment, processing of the extraction step S220 is executed next to the prediction equation generation step S210.

In the extraction step S220, the extraction unit 220 extracts the sensors 10 having a high degree of contribution to the prediction result in the prediction equation based on the weights in the prediction equation and a predetermined condition regarding the weights. Specifically, the extraction unit 220 acquires information indicating the prediction equation from the prediction equation generation unit 210. Then, the magnitude of the weight to the feature value of each sensor 10 indicated in information indicating the prediction equation is computed.

Here, WX in the prediction equation z=WX+b can be rewritten as w₁x₁+w₂x₂+ . . . w_{J}x_{J} using the feature value xⱼ based on the time-series data 14 of each sensor 10 included in the set 100 and a weight wⱼ to the feature value xⱼ. Note that wⱼ may be a numerical value or may be a vector. In a case where wⱼ is a vector, each element of wⱼ is a weight to each feature value that is an element of xⱼ. Then, the magnitude of the weight is, for example, a norm of wⱼ. On the other hand, in a case where wⱼ is a numerical value, the magnitude of the weight is an absolute value of wⱼ.

The extraction unit 220 further determines whether or not the computed magnitude of the weight satisfies a predetermined condition. Information indicating the condition is stored in advance in a storage apparatus accessible from the extraction unit 220. For example, in a case where the condition indicates a condition regarding the sensor 10 having a high degree of contribution to the prediction result, such as “the magnitude of the weight is equal to or greater than a reference value”, the extraction unit 220 extracts the sensor 10 corresponding the weight satisfying the condition. On the other hand, in a case where the condition indicates a condition of the sensor 10 having low contribution to the prediction result, such as “the magnitude of the weight is equal to or less than a reference value”, the extraction unit 220 extracts the sensor 10 corresponding to the weight not satisfying the condition. Then, the extraction unit 220 generates combination information indicating a combination including the extracted sensors 10. Information indicating the prediction equation is associated with the generated combination information.

Note that, in the time-series data 14 shown in FIG. 3, information regarding the molecules absorbed and detached to and from the sensor 10 is considered to be strongly reflected in a portion where the output significantly fluctuates at the head of each of the period P1 and the period P2. Accordingly, the weight of the feature value based on data of the head portion is predicted to be large. Then, in a case where the weight of the feature value based on data of a steady portion in the period P1 and the period P2 is large, the results is considered to be affected by noise or the like. Therefore, the extraction unit 220 may extract the sensors 10 based on the weight to the feature value based on only data of a part of the period P1 and the period P2. Specifically, the sensors 10 may be extracted based on the weight to the feature value based on data until a predetermined time from the start of the period in each of the period P1 and the period P2.

Next, in the output step S250, the output unit 250 outputs at least one of the sensors 10 having a high degree of contribution to the prediction result and the sensors 10 having a low degree of contribution to the prediction result based on an extraction result for the sensors 10. Note that the output of the sensors 10 from the output unit 250 is an output of symbols or the like indicating the sensors 10. Note that, in a case where the output unit 250 outputs both the sensors 10 having a high degree of contribution to the prediction result and the sensors 10 having a low degree of contribution to the prediction result, the sensors 10 are output in an identifiable state from each other. Alternatively, the output unit 250 may further output information indicating the prediction equation.

Specifically, display indicating at least one of the sensors 10 having a high degree of contribution to the prediction result and the sensors 10 having a low degree of contribution to the prediction result is displayed on a display apparatus provided in the processing apparatus 20. Alternatively, the output unit 250 may output information indicating at least one of the sensors 10 having a high degree of contribution to the prediction result and the sensors 10 having a low degree of contribution to the prediction result to an external apparatus or may store the above-described information in the storage apparatus accessible from the output unit 250.

Even in the example embodiment, the user can search for a combination of the sensors 10 to be employed using the output of the output unit 250 in the same manner as in the first example embodiment.

The processing apparatus 20 according to the example embodiment can also be implemented by the computer 1000 shown in FIG. 6. In the example embodiment, the storage device 1080 further stores a program module that implements the extraction unit 220 of the processing apparatus 20.

Next, the operations and effects of the example embodiment will be described. In the example embodiment, the same operations and effects as in the first example embodiment are obtained. In addition, with the processing apparatus 20 according to the example embodiment, the sensors 10 having a low or high degree of contribution to the prediction result can be recognized based on an extraction result of the extraction unit 220. Consequently, an appropriate combination of the sensors can be clearly recognized to obtain sensors according to a desired purpose.

### Third Example Embodiment

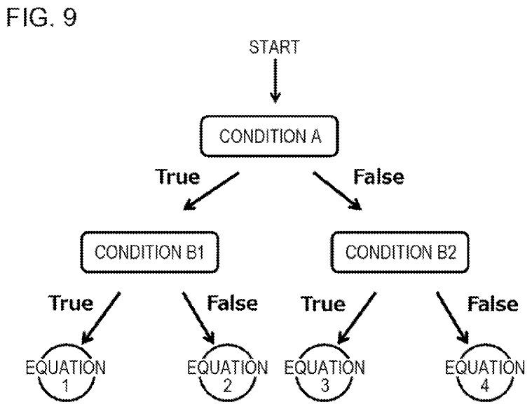

FIG. 9 is a diagram illustrating a prediction model that is used in machine learning to be performed by a prediction equation generation unit 210 according to a third example embodiment. A processing apparatus 20 according to the example embodiment is the same as the processing apparatus 20 according to the second example embodiment except for the following matters.

In the processing apparatus 20 according to the example embodiment, the prediction equation generation unit 210 generates a prediction equation using a model including a branch based on a detection environment of the sensor 10. Furthermore, the output unit 250 outputs a condition of the detection environment appropriate for the prediction equation and based on a condition of the branch in association with information indicating the prediction equation.

An output of the sensor 10 changes depending on the detection environment, that is, a measurement condition, not only on the components of the target gas. Accordingly, a preferable combination of the sensors 10 may be different for each detection environment. In the example embodiment, the prediction equation generation unit 210 can derive a preferable combination of the sensors 10 corresponding to the detection environment by generating the prediction equation using the model including the branch based on the detection environment.

The detection environment is not particularly limited, for example, and includes, for example, at least one of a temperature, humidity, atmospheric pressure, a kind of impure gas, a kind of purge gas, a sampling period of an odor component, a distance between a target and the sensor 10, and an object present around the sensor 10. The temperature, the humidity, and the atmospheric pressure are a temperature, humidity, and atmospheric pressure around the sensor 10, respective, and specifically, a temperature, humidity, and atmospheric pressure of an atmosphere surrounding a functional part of the sensor 10. The kind of the impure gas is a kind of gas that is supplied to the sensor 10 along with a target odor component in the operation to expose the sensor 10 to the target gas. Specifically, examples of the kind of the impure gas include inert gas, such as nitrogen, air, and the like. The kind of the purge gas is gas that is supplied to the sensor 10 in the operation to remove the gas to be measured from the sensor 10. Specifically, examples of the purge gas include inert gas, such as nitrogen, air, and the like. The sampling period of the odor component is a repetition period in a case where the operation to expose the sensor 10 to the gas to be measured and the operation to remove the gas to be measured from the sensor 10 are repeatedly performed. The distance between the target and the sensor 10 is a distance between a specific target and the sensor 10 in a case where the sensor 10 is disposed around the target to perform detection. The object that is present around the sensor 10 is a kind of a target in a case where the sensor 10 is disposed around the specific target to perform detection.

The model that is used in the machine learning has a hierarchical structure specifically including a plurality of nodes. Then, a branch equation is positioned as a condition of a branch on one or more intermediate nodes, and a prediction equation is positioned on an anode in a lowest layer. In the drawing, a condition A, a condition B1, and a condition B2 are conditions of branches, and Equation 1 to Equation 4 are prediction equations. Note that a specific configuration of the model, such as the number of intermediate nodes or the number of anodes, is not particularly limited.

In the example embodiment, the machine learning that is performed by the prediction equation generation unit 210 is, for example, heterogeneous mixture learning that further has the detection environment of the sensor 10 as an input. Here, the detection environment is associated with the feature value as the input of the machine learning, and is a detection environment when the time-series data 14 that is a source of the feature value is obtained. With the heterogeneous mixture learning, a specific model including a condition of a branch is generated along with a prediction equation.

In the example embodiment, the prediction equation generation unit 210 performs machine learning having a plurality of learning data sets obtained in a plurality of detection environments in the prediction equation generation step S210 as inputs. As described above, each learning data set includes a plurality of feature values obtained by the set 100 of the sensors 10 and correct answer data. Then, one or more prediction equations are generated as a result of the machine learning.

Here, a condition of a detection environment to be a premise is linked with each prediction equation. Each prediction equation is particularly valid in an environment satisfying a condition of the detection environment associated with the prediction equation. The condition of the detection environment is based on a branch condition in a model generated simultaneously with the prediction equation. In detail, the condition of the detection environment is determined by branch conditions from a start to a prediction equation on an anode in the generated model and determination results of the branch conditions. For example, in the example of the drawing, in a case where the condition A is that “temperature>T₁”, the condition B2 is that “humidity>H₁”, a condition of a detection environment associated with Equation 3 is that “temperature is equal to or lower than T₁ and humidity is higher than H₁”.

Next, the extraction step S220 is executed by the extraction unit 220. In the extraction step S220 according to the example embodiment, a combination of the sensors 10 that are suitably usable in a specific usage environment selected by the user is extracted. The processing apparatus 20 can receive an input from the user, for example, and the extraction unit 220 acquires information indicating the usage environment input by the user. Note that information indicating the usage environment may be determined in advance and may be held in the storage apparatus accessible from the extraction unit 220. Information indicating the usage environment is, for example, one or more of a temperature, humidity, atmospheric pressure, a kind of impure gas, a kind of purge gas, a sampling period of an odor component, a distance between a target and the sensor 10, and an object present around the sensor 10.

Then, the extraction unit 220 selects a prediction equation corresponding to the condition of the detection environment satisfied by the usage environment from among a plurality of prediction equations generated by the prediction equation generation unit 210. Moreover, the extraction unit 220 extracts the sensors 10 for the selected prediction equation in the same manner as described in the second example embodiment, and generates combination information. Information indicating the condition of the detection environment is further associated with the combination information.

The output unit 250 executes the same processing as the processing of the output step S250 described in the second example embodiment in the output step S250. Note that the output unit 250 may further output the condition of the detection environment associated with the prediction equation.

Note that, in a case where the usage environment satisfies the condition of the detection environment regarding a plurality of prediction equations, the extraction unit 220 may select a plurality of prediction equations and may generate combination information for each prediction equation. Alternatively, the output unit 250 may output a plurality of combinations. Note that the output unit 250 outputs information indicating the prediction equation, or the like in an identifiable state for each combination.

Note that the specific model including the condition of the branch to be used in the machine learning may be set by the user instead of being generated by the machine learning. In this case, the machine learning may not be the heterogeneous mixture learning.

In the heterogeneous mixture learning, although the branch condition is repeatedly updated along with the prediction equation during the repetition of learning, a model obtained in the middle of learning may be fixedly used in subsequent learning.

The processing apparatus 20 according to the example embodiment may not include the extraction unit 220 like the processing apparatus 20 according to the first example embodiment. In this case, the output unit 250 outputs information indicating one or more prediction equations generated by the prediction equation generation unit 210.

The extraction unit 220 may generate combination information for all prediction equations generated by the prediction equation generation unit 210, and the output unit 250 may output the sensors 10, information indicating the prediction equations, and the condition of the detection environment regarding all the generated combination information. In this case, the user can comprehensively view the output information to determine a preferable combination of the sensors 10 in all conditions of a plurality of detection environments. For example, the user can exclude the sensors 10, which are not included in any combinations, from the candidates of the sensors 10 to be used. Alternatively, only the sensors 10 that are included in all combinations can be left as the candidates. Furthermore, the sensors 10 that are included only in a combination associated with an extreme condition hard to suppose for practical use as the condition of the detection environment can be excluded from candidates.

Next, the operations and effects of the example embodiment will be described. In the example embodiment, the same operations and effects as in the first example embodiment are obtained. In addition, the prediction equation generation unit 210 can derive a preferable combination of the sensors 10 corresponding to the detection environment by generating the prediction equation using the model including the branch based on the detection environment.

### Fourth Example Embodiment

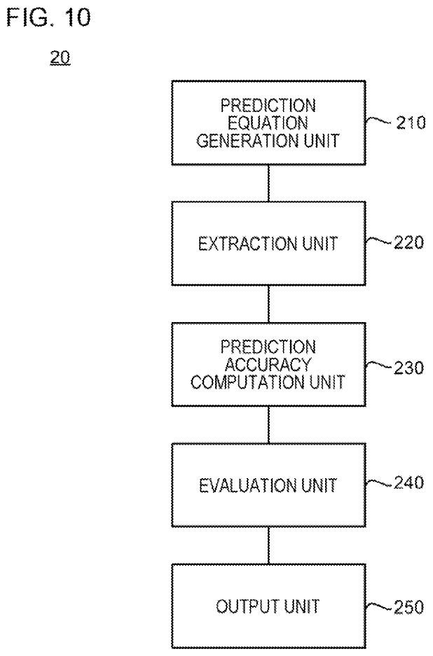

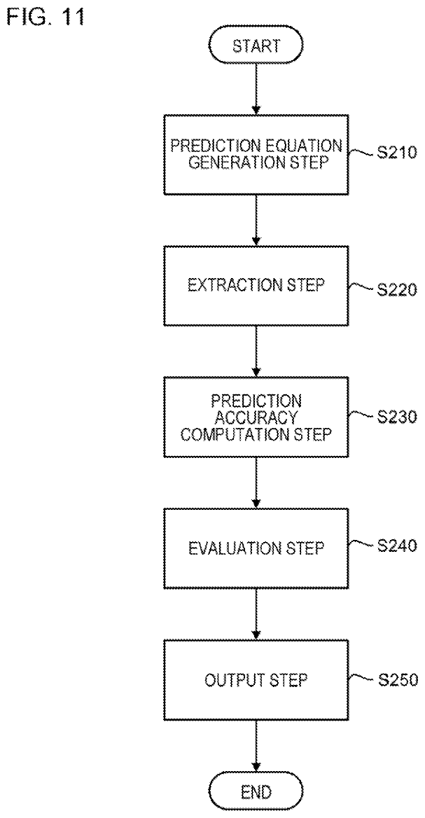

FIG. 10 is a diagram illustrating the configuration of a processing apparatus 20 according to a fourth example embodiment. FIG. 11 is a flowchart illustrating a processing method according to a fourth example embodiment. The processing apparatus 20 according to the example embodiment is the same as the processing apparatus 20 according to at least one of the second and third example embodiments except for the following matters.

In an example of FIG. 10, the processing apparatus 20 further includes a prediction accuracy computation unit 230 that computes prediction accuracy of the prediction equation, and an evaluation unit 240 that evaluates a combination of the sensors 10. In an example of FIG. 11, the processing method further includes a prediction accuracy computation step S230 and an evaluation step S240. Note that the processing apparatus 20 according to the example embodiment may not include at least one of the prediction accuracy computation unit 230 and the evaluation unit 240. Furthermore, the processing method according to the example embodiment may not include at least one of the prediction accuracy computation step S230 and the evaluation step S240.

In the prediction equation generation step S210 of the example embodiment, the same processing as in the prediction equation generation step S210 according to at least one of the first to third example embodiments is executed. Next, in the extraction step S220 of the example embodiment, the same processing as in the extraction step S220 according to at least one of the second and third example embodiments is executed.

In the processing apparatus 20 according to the example embodiment, next to the extraction step S220, processing of the prediction accuracy computation step S230 is executed by the prediction accuracy computation unit 230. Note that a timing at which the processing of the prediction accuracy computation step S230 is executed is not particularly limited as long as the timing is after the prediction equation generation step S210 and before the evaluation step S240 described below. Note that, in a case where the processing apparatus 20 does not include the evaluation unit 240, the timing at which the processing of the prediction accuracy computation step S230 is executed may be after the prediction equation generation step S210 and before the output step S250.

In the prediction accuracy computation step S230, the prediction accuracy computation unit 230 computes the prediction accuracy of each prediction equation. In the computation of the prediction accuracy, the same data set as the learning data set is used as an evaluation data set. That is, the evaluation data set includes a plurality of feature values and correct answer data.

Note that the completely same data set is not included in a plurality of learning data sets and a plurality of evaluation data sets. For example, a part of a plurality of different data sets generated outside or inside the processing apparatus 20 can be used as a plurality of learning data sets, and the remaining data sets can be used as a plurality of evaluation data sets.

The prediction accuracy is regression accuracy with respect to prediction based on regression, and is, for example, a least-square error or a root mean-square error (RMSE). The prediction accuracy is discrimination accuracy with respect to prediction based on discrimination, and is, for example, a precision ratio, a recall ratio, an F-value, a correct answer ratio, or ROC_AUC.

An example of a method in which the prediction accuracy computation unit 230 computes the prediction accuracy will be described in detail. The prediction accuracy computation unit 230 can acquire or generate a plurality of evaluation data sets by the same method as the method in which the prediction equation generation unit 210 acquires or generates the learning data set. The prediction accuracy computation unit 230 inputs the feature values included in the evaluation data set to the prediction equation of which the accuracy is attempted to be evaluated, thereby obtaining a prediction result. Then, determination is made whether or not the obtained prediction result coincides with the correct answer data included in the evaluation data set. Then, the prediction accuracy computation unit 230 executes the same processing on a plurality of evaluation data sets and computes a probability that the prediction result coincides with the correct answer data, as the prediction accuracy of the prediction equation. The computed prediction accuracy is associated with the prediction equation.

A plurality of evaluation data sets may be based on measurement results in different detection environments. Note that, as in the first or second example embodiment, in a case where one prediction equation is generated for one set 100, it is preferable that the evaluation data set is data obtained in a detection environment similar to a detection environment in which the learning data set is obtained. As in the third example embodiment, in a case where a plurality of prediction equations are generated for one set 100, regarding each prediction equation, only the evaluation data set obtained in an environment satisfying the condition of the detection environment associated with the prediction equation is used in the computation of the prediction accuracy.

Next, processing of the evaluation step S240 is executed by the evaluation unit 240. The evaluation unit 240 evaluates the combination of the sensors 10 based on at least one of, for example, the prediction accuracy of the prediction equation to be used in a case where the combination is employed and a cost in a case where the combination is employed. Above all, it is preferable that the evaluation unit 240 evaluates the combination of the sensors 10 based on at least the cost in a case where the combination of the sensors 10 indicated in the combination information is employed.

The cost includes, for example, an initial cost and a running cost. Examples of the initial cost include a manufacturing cost and a procurement cost of the sensor 10. Examples of the running cost include a management cost, a replacement cost due to deterioration or the like of the sensor 10, and human labor in handling.

A parameter indicating the cost of each sensor 10 is held in advance in a storage apparatus accessible by the evaluation unit 240, and the evaluation unit 240 acquires the parameters indicating the costs of the sensors 10 included in the combination from the storage apparatus. Then, the parameters indicating the costs regarding all sensors 10 included in the combination are added to obtain a sum.

The evaluation unit 240 acquires the prediction accuracy of the prediction equation associated with the combination information from the prediction accuracy computation unit 230.

The evaluation unit 240 evaluates the combination further using an evaluation function. The evaluation function is a function that computes an evaluation value based on one or more factors. Specifically, the evaluation function is represented by a linear sum of an evaluation parameter indicating an evaluation result in each factor. For example, an evaluation parameter with a cost as a factor is the sum computed in the above-described manner, and an evaluation parameter with accuracy as a factor is the prediction accuracy acquired from the prediction accuracy computation unit 230. In the evaluation function, each evaluation parameter is multiplied by a coefficient, and a weight of each factor to the evaluation result is balanced or directivity of evaluation is determined. The coefficient is determined for each kind of the evaluation parameter.

The evaluation unit 240 computes an evaluation value as an evaluation result by applying the sum of the parameter indicating the cost and the prediction accuracy to the evaluation function. Note that the evaluation result obtained by the evaluation unit 240 is higher as the sum regarding the cost is smaller and is higher as the prediction accuracy is better. Information indicating the evaluation function is held in advance in the storage apparatus accessible by the evaluation unit 240. The computed evaluation value is associated with the combination information.

The evaluation unit 240 may evaluate the combination of the sensors 10 further based on the number of sensors 10 included in the combination. For example, in a case where the number of sensors 10 included in the combination is set as a factor, for example, the number of sensors 10 can be an evaluation parameter in the evaluation function. Note that the evaluation result obtained by the evaluation unit 240 is higher as the number of sensors 10 included in the combination is smaller.

As in the third example embodiment, in a case where a plurality of prediction equations are generated for one set 100, the evaluation unit 240 may evaluate the combination of the sensors 10 further based on the condition of the detection environment associated with the combination information. For example, in a case where the extent of the condition of the detection environment is set as a factor, for example, a width of a range of the temperature, the humidity, the atmospheric pressure, the period, the distance, or the like indicated as the condition of the detection environment, or the number of options of gas or the object can be an evaluation parameter in the evaluation function. Furthermore, in a case where practicability of the condition of the detection environment is set as a factor, a distance between a central value of a range of the temperature, the humidity, the atmospheric pressure, the period, the distance, or the like indicated as the condition of the detection environment and a predetermined standard value can be an evaluation parameter in the evaluation function. That is, it can be said that the smaller the distance is, the higher the practicability is. Note that the evaluation result obtained by the evaluation unit 240 is higher as the extent of the condition of the detection environment is higher and is higher as the practicability of the condition of the detection environment is higher.

In the output step S250, the output unit 250 further outputs the evaluation result computed by the evaluation unit 240 in association with the combination of the sensors 10. The user can compare a plurality of combinations of the sensors 10 using the evaluation result. For example, in a case where the processing by the processing apparatus 20 is repeated while changing the configuration of the set 100, results for the respective sets 100 can be compared based on the evaluation result, and a most excellent combination of the sensors 10 can be derived. As in the third example embodiment, in a case where a plurality of pieces of combination information are generated based on one set 100, the combination information can be compared based on the evaluation result. Note that the output unit 250 may output the prediction accuracy of the prediction equation in addition to the evaluation result or instead of the evaluation result.

In addition, as described in the third example embodiment, for example, a case where the user comprehensively views the output information to attempt to determine a preferable combination of the sensors 10 in all the conditions of a plurality of pieces of detection environment will be described. In this case, for example, the sensors 10 of the set 100 are rearranged such that the evaluation values to a plurality of pieces of combination information generated for the set 100 exceed a predetermined threshold value, and an average value of the evaluation values increases. In this manner, a combination of the sensors 10 appropriate for a purpose is obtained as the set 100. The rearrangement of the set 100 can be manually performed by the user. Note that, in a case where the learning data set and the evaluation data set are obtained by simulations, the rearrangement of the set 100 may be virtually performed by a simulation apparatus.

In the processing apparatus 20 according to the example embodiment, the evaluation results may be compared for combinations based on a plurality of sets 100. For example, the prediction equation generation unit 210 performs machine learning regarding each of a plurality of sets 100. The extraction unit 220 generates combination information for each of a plurality of sets 100. Then, the evaluation unit 240 evaluates each of a plurality of combinations indicated by a plurality of pieces of generated combination information. The output unit 250 outputs a combination having a most excellent (high) evaluation result by the evaluation unit 240 among a plurality of combinations. Note that the output unit 250 may output a plurality of combinations in a state in which the combination having the most excellent evaluation result is identifiable.

The processing apparatus 20 according to the example embodiment can also be implemented by the computer 1000 shown in FIG. 6. In the example embodiment, the storage device 1080 further stores program modules that implement the prediction accuracy computation unit 230 and the evaluation unit 240 of the processing apparatus 20, respectively.

Next, the operations and effects of the example embodiment will be described. In the example embodiment, the same operations and effects as in the first example embodiment are obtained. In addition, the prediction accuracy of the prediction equation is computed by the prediction accuracy computation unit 230 or the evaluation by the evaluation unit 240 is performed, whereby the validity of a plurality of combinations of the sensors 10 can be compared.

Although the example embodiments of the invention have been described referring to the drawings, the example embodiment is merely an example of the invention. The invention can employ various configurations other than the above. For example, in the sequence diagrams or the flowcharts used in the above description, although a plurality of steps (processing) are described in order, an execution order of the steps executed in the respective example embodiments is not limited to the described order. In the respective example embodiments, an order of the steps shown in the drawings can be changed within a range without hindrance in contents. Furthermore, the respective example embodiments described above can be combined within a range in which the contents do not conflict with each other.

Although the example embodiments of the invention have been described referring to the drawings, the example embodiment is merely an example of the invention. The invention can employ various configurations other than the above.

A part or the whole of the above-described example embodiments can be described as, but is not limited to, the following supplementary notes.

1-1. A processing apparatus including:
a prediction equation generation unit that generates, through machine learning having a plurality of feature values based on outputs from a set of a plurality of kinds of sensors and correct answer data as inputs, a prediction equation that has the plurality of feature values as variables and is used for predicting an odor component;
an extraction unit that extracts one or more sensors from the set based on a plurality of weights to the plurality of feature values in the prediction equation; and
an output unit that outputs at least one of the sensors extracted by the extraction unit and the unextracted sensors in an identifiable state,
in which the extraction unit extracts the sensors that are output sources of the feature values weighted with the weights satisfying or not satisfying a predetermined condition among the plurality of weights in the prediction equation.

1-2. A processing apparatus including:
a prediction equation generation unit that generates, through machine learning having a plurality of feature values based on outputs from a set of a plurality of kinds of sensors and correct answer data as inputs, a prediction equation that has the plurality of feature values as variables and is used for predicting an odor component; and
an output unit that outputs a plurality of weights to the plurality of feature values in the prediction equation as information indicating the prediction equation in association with the feature values, respectively.

1-3. The processing apparatus described in 1-2, further comprising:
an extraction unit that extracts one or more sensors from the set based on a plurality of weights to the plurality of feature values in the prediction equation,
in which the extraction unit extracts the sensors that are output sources of the feature values weighted with the weights satisfying or not satisfying a predetermined condition among the plurality of weights in the prediction equation.

1-4. The processing apparatus described in 1-1 or 1-3,
in which the extraction unit generates combination information indicating a combination including the extracted sensors, and
the processing apparatus further includes:
an evaluation unit that evaluates the combination based on at least a cost in a case where the combination is employed.

1-5. The processing apparatus described in 1-4,
in which the prediction equation generation unit performs the machine learning on each of a plurality of the sets,
the extraction unit generates the combination information for each of the plurality of sets,
the evaluation unit evaluates each of a plurality of the combinations indicated by a plurality of pieces of the generated combination information, and
the output unit outputs the combination having a most excellent evaluation result by the evaluation unit among the plurality of combinations.

1-6. The processing apparatus described in any one of 1-1 to 1-5,
in which the prediction equation generation unit generates the prediction equation using a model including branches based on detection environments of the sensors, and
the output unit further outputs a condition of the detection environment appropriate for the prediction equation and based on a condition of the branch in association with information indicating the prediction equation.

1-7. The processing apparatus described in 1-6,
in which the machine learning is heterogeneous mixture learning further having the detection environments of the sensors associated with the feature values as an input, and
the condition of the branch is generated by the heterogeneous mixture learning.

1-8. The processing apparatus described in 1-6 or 1-7,
in which the detection environment includes at least one of a temperature, humidity, atmospheric pressure, a kind of impure gas, a kind of purge gas, a sampling period of the odor component, a distance between a target and the sensor, and an object present around the sensor.

1-9. The processing apparatus described in any one of 1-1 to 1-8, further including:
a prediction accuracy computation unit that computes prediction accuracy of the prediction equation.

2-1. A processing method including:
a prediction equation generation step of generating, through machine learning having a plurality of feature values based on outputs from a set of a plurality of kinds of sensors and correct answer data as inputs, a prediction equation that has the plurality of feature values as variables and is used for predicting an odor component;
an extraction step of extracting one or more sensors from the set based on a plurality of weights to the plurality of feature values in the prediction equation; and
an output step of outputting at least one of the sensors extracted in the extraction step and the unextracted sensors in an identifiable state,
in which, in the extraction step, the sensors that are output sources of the feature values weighted with the weights satisfying or not satisfying a predetermined condition among the plurality of weights in the prediction equation are extracted.

2-2. A processing method including:
a prediction equation generation step of generating, through machine learning having a plurality of feature values based on outputs from a set of a plurality of kinds of sensors and correct answer data as inputs, a prediction equation that has the plurality of feature values as variables and is used for predicting an odor component; and
an output step of outputting a plurality of weights to the plurality of feature values in the prediction equation as information indicating the prediction equation in association with the feature values, respectively.

2-3. The processing method described in 2-2., further comprising:
an extraction step of extracting one or more sensors from the set based on a plurality of weights to the plurality of feature values in the prediction equation,
in which, in the extraction step, the sensors that are output sources of the feature values weighted with the weights satisfying or not satisfying a predetermined condition among the plurality of weights in the prediction equation are extracted.

2-4. The processing method described in 2-1 or 2-3,
in which, in the extraction step, combination information indicating a combination including the extracted sensors is generated, and
the processing method further includes:
an evaluation step of evaluating the combination based on at least a cost in a case where the combination is employed.

2-5. The processing method described in 2-4,
in which, in the prediction equation generation step, the machine learning is performed on each of a plurality of the sets,
in the extraction step, the combination information is generated for each of the plurality of sets,
in the evaluation step, each of a plurality of the combinations indicated by a plurality of pieces of the generated combination information is evaluated, and
in the output step, the combination having a most excellent evaluation result in the evaluation step among the plurality of combinations is further output.

2-6. The processing method described in any one of 2-1 to 2-5,
in which, in the prediction equation generation step, the prediction equation is generated using a model including branches based on detection environments of the sensors, and
in the output step, a condition of the detection environment appropriate for the prediction equation and based on a condition of the branch is further output in association with information indicating the prediction equation.

2-7. The processing method described in 2-6,
in which the machine learning is heterogeneous mixture learning further having the detection environments of the sensors associated with the feature values as an input, and
the condition of the branch is generated by the heterogeneous mixture learning.

2-8. The processing method described in 2-6 or 2-7,
in which the detection environment includes at least one of a temperature, humidity, atmospheric pressure, a kind of impure gas, a kind of purge gas, a sampling period of the odor component, a distance between a target and the sensor, and an object present around the sensor.

2-9. The processing method described in any one of 2-1 to 2-8, further including:
a prediction accuracy computation step of computing prediction accuracy of the prediction equation.

3-1. A program causing a computer each step of the processing method described in any one of 2-1 to 2-9.

What is claimed is:

1. A processing apparatus comprising:
a prediction equation generation unit that generates, through machine learning having a plurality of feature values based on outputs from a set of a plurality of kinds of sensors and correct answer data as inputs, a prediction equation that has the plurality of feature values as variables and is used for predicting an odor component;
an extraction unit that extracts one or more sensors from the set based on a plurality of weights to the plurality of feature values in the prediction equation; and
an output unit that outputs at least one of the sensors extracted by the extraction unit and the unextracted sensors in an identifiable state,
wherein the extraction unit extracts the sensors that are output sources of the feature values weighted with the weights satisfying or not satisfying a predetermined condition among the plurality of weights in the prediction equation.

2. A processing apparatus comprising:
a prediction equation generation unit that generates, through machine learning having a plurality of feature values based on outputs from a set of a plurality of kinds of sensors and correct answer data as inputs, a prediction equation that has the plurality of feature values as variables and is used for predicting an odor component; and
an output unit that outputs a plurality of weights to the plurality of feature values in the prediction equation as information indicating the prediction equation in association with the feature values, respectively.

3. The processing apparatus according to claim 2, further comprising:
an extraction unit that extracts one or more sensors from the set based on a plurality of weights to the plurality of feature values in the prediction equation,
wherein the extraction unit extracts the sensors that are output sources of the feature values weighted with the weights satisfying or not satisfying a predetermined condition among the plurality of weights in the prediction equation.

4. The processing apparatus according to claim 1,
wherein the extraction unit generates combination information indicating a combination including the extracted sensors, and
the processing apparatus further comprises:
an evaluation unit that evaluates the combination based on at least a cost in a case where the combination is employed.

5. The processing apparatus according to claim 4,
wherein the prediction equation generation unit performs the machine learning on each of a plurality of the sets,
the extraction unit generates the combination information for each of the plurality of sets,
the evaluation unit evaluates each of a plurality of the combinations indicated by a plurality of pieces of the generated combination information, and
the output unit outputs the combination having a most excellent evaluation result by the evaluation unit among the plurality of combinations.

6. The processing apparatus according to claim 1,
wherein the prediction equation generation unit generates the prediction equation using a model including branches based on detection environments of the sensors, and
the output unit further outputs a condition of the detection environment appropriate for the prediction equation and based on a condition of the branch in association with information indicating the prediction equation.

7. The processing apparatus according to claim 6,
wherein the machine learning is heterogeneous mixture learning further having the detection environments of the sensors associated with the feature values as an input, and
the condition of the branch is generated by the heterogeneous mixture learning.

8. The processing apparatus according to claim 6,
wherein the detection environment includes at least one of a temperature, humidity, atmospheric pressure, a kind of impure gas, a kind of purge gas, a sampling period of the odor component, a distance between a target and the sensor, and an object present around the sensor.

9. The processing apparatus according to claim 1, further comprising:
a prediction accuracy computation unit that computes prediction accuracy of the prediction equation.

10. A processing method comprising:
generating, through machine learning having a plurality of feature values based on outputs from a set of a plurality of kinds of sensors and correct answer data as inputs, a prediction equation that has the plurality of feature values as variables and is used for predicting an odor component;
extracting one or more sensors from the set based on a plurality of weights to the plurality of feature values in the prediction equation; and
outputting at least one of the sensors extracted and the unextracted sensors in an identifiable state,
wherein, the sensors that are output sources of the feature values weighted with the weights satisfying or not satisfying a predetermined condition among the plurality of weights in the prediction equation are extracted.

11. A processing method comprising:
generating, through machine learning having a plurality of feature values based on outputs from a set of a plurality of kinds of sensors and correct answer data as inputs, a prediction equation that has the plurality of feature values as variables and is used for predicting an odor component; and
outputting a plurality of weights to the plurality of feature values in the prediction equation as information indicating the prediction equation in association with the feature values, respectively.

12. The processing method according to claim 11, further comprising:
extracting one or more sensors from the set based on a plurality of weights to the plurality of feature values in the prediction equation,
wherein, the sensors that are output sources of the feature values weighted with the weights satisfying or not satisfying a predetermined condition among the plurality of weights in the prediction equation are extracted.

13. The processing method according to claim 10,
wherein, combination information indicating a combination including the extracted sensors is generated, and
the processing method further comprises:
evaluating the combination based on at least a cost in a case where the combination is employed.

14. The processing method according to claim 13,
wherein, the machine learning is performed on each of a plurality of the sets,
the combination information is generated for each of the plurality of sets,
each of a plurality of the combinations indicated by a plurality of pieces of the generated combination information is evaluated, and
the combination having a most excellent evaluation result among the plurality of combinations is further output.

15. The processing method according to claim 10,
wherein, the prediction equation is generated using a model including branches based on detection environments of the sensors, and
a condition of the detection environment appropriate for the prediction equation and based on a condition of the branch is further output in association with information indicating the prediction equation.

16. The processing method according to claim 15,
wherein the machine learning is heterogeneous mixture learning further having the detection environments of the sensors associated with the feature values as an input, and
the condition of the branch is generated by the heterogeneous mixture learning.

17. The processing method according to claim 15,
wherein the detection environment includes at least one of a temperature, humidity, atmospheric pressure, a kind of impure gas, a kind of purge gas, a sampling period of the odor component, a distance between a target and the sensor, and an object present around the sensor.

18. The processing method according to claim 10, further comprising:
computing prediction accuracy of the prediction equation.

19. A non-transitory storage medium storing a program causing a computer to execute a processing method, the processing method comprising:
generating, through machine learning having a plurality of feature values based on outputs from a set of a plurality of kinds of sensors and correct answer data as inputs, a prediction equation that has the plurality of feature values as variables and is used for predicting an odor component;
extracting one or more sensors from the set based on a plurality of weights to the plurality of feature values in the prediction equation; and
outputting at least one of the sensors extracted and the unextracted sensors in an identifiable state,
wherein, the sensors that are output sources of the feature values weighted with the weights satisfying or not satisfying a predetermined condition among the plurality of weights in the prediction equation are extracted.

20. The processing apparatus according to claim 2,
wherein the prediction equation generation unit generates the prediction equation using a model including branches based on detection environments of the sensors, and
the output unit further outputs a condition of the detection environment appropriate for the prediction equation and based on a condition of the branch in association with information indicating the prediction equation.

21. The processing apparatus according to claim 2, further comprising:
a prediction accuracy computation unit that computes prediction accuracy of the prediction equation.

22. The processing method according to claim 11,
wherein, the prediction equation is generated using a model including branches based on detection environments of the sensors, and
a condition of the detection environment appropriate for the prediction equation and based on a condition of the branch is further output in association with information indicating the prediction equation.

23. The processing method according to claim 11, further comprising:
computing prediction accuracy of the prediction equation.

24. A non-transitory storage medium storing a program causing a computer to execute a processing method, the processing method comprising:
generating, through machine learning having a plurality of feature values based on outputs from a set of a plurality of kinds of sensors and correct answer data as inputs, a prediction equation that has the plurality of feature values as variables and is used for predicting an odor component; and
outputting a plurality of weights to the plurality of feature values in the prediction equation as information indicating the prediction equation in association with the feature values, respectively.
